(19) United States
(12) Patent Application Publication
(10) Pub. No.: US 2024/0091474 A1
(43) Pub. Date: Mar. 21, 2024

(54) SAFE VENTILATION IN THE PRESENCE OF RESPIRATORY EFFORT

(71) Applicant: KONINKLIJKE PHILIPS N.V., Eindhoven (NL)

(72) Inventors: Cornelis Petrus Hendriks, Eindhoven (NL); Roberto Buizza, Eindhoven (NL); Jaap Roger Haartsen, Eindhoven (NL); Joerg Sabczynski, Hamburg (DE); Thomas Koehler, Hamburg (DE); Rafael Wiemker, Hamburg (DE); Michael Polkey, London (GB)

(21) Appl. No.: 18/219,231

(22) Filed: Jul. 7, 2023

Related U.S. Application Data
(60) Provisional application No. 63/407,772, filed on Sep. 19, 2022.

(51) Int. Cl. A61M 16/00; G06T 7/00

(52) U.S. Cl. CPC A61M 16/024; A61M 16/0051; G06T 7/0012; G06T 2207/10081

(57) ABSTRACT

A mechanical ventilation device includes at least one electronic controller configured to receive imaging data related to a dimension of a diaphragm of a patient during inspiration and expiration while the patient undergoes mechanical ventilation therapy with an associated mechanical ventilator; calculate a pressure value (Pₗ, DPₗ) of a chest of the patient based on at least the imaging data; and when the calculated pressure value (Pₗ, DPₗ) does not satisfy an acceptance criterion, at least one of output an alert indicative of the calculated pressure value (Pₗ, DPₗ) failing to satisfy the acceptance criterion; and output a recommended adjustment to one or more parameters of the mechanical ventilation therapy delivered to the patient.

### CROSS-REFERENCE TO RELATED APPLICATIONS

This patent application claims the priority benefit under 35 U.S.C. § 119(e) of U.S. Provisional Application No. 63/407,772, filed on Sep. 19, 2022, the contents of which are herein incorporated by reference.

The following relates generally to the respiratory therapy arts, mechanical ventilation arts, mechanical ventilation weaning arts, ventilator induced lung injury (VILI) arts, and related arts.

### BACKGROUND

Clinicians can use pressure-volume (P-V) loops and parameter estimations of the respiratory system (e.g., resistance and compliance) to monitor the patient status and to offer safe ventilation therapy to the patient. Most accurate results are obtained using the transpulmonary pressure, which is accessible with esophageal catheter measurements. However, often a catheter is not present, in which case the above estimations are impacted by the presence of a patient effort and the chest wall mechanics. Modern ventilators, therefore, avoid a patient effort and ignore chest wall mechanics when determining the transpulmonary pressure.

Respiratory therapists can use a P-V loop as guidance for choosing the right ventilator settings to avoid atelectasis and overdistension (VILI). Ventilators can display P-V loops on their screen. Ventilators should use the transpulmonary pressure to construct the P-V loop accurately, since the transpulmonary pressure is responsible for the deformation of lung tissue, (i.e., the parenchyma tissue with alveoli). The transpulmonary pressure Pₗ is the difference between a pleural pressure and an alveolar pressure.

However, the transpulmonary pressure is difficult to measure as state-of-the-art requires the use of catheters (see, e.g., Umbrello, M. and Chiumello, D., 2018, “Interpretation of the transpulmonary pressure in the critically ill patient”, Ann Transl Med 2018; 6(19):383). For that reason, ventilators use the measured pressure at the airway opening, P_{wye}, and the derived alveolar pressure Pₐₗᵥ, to construct the P-V loop. This method is accurate if there is no respiratory effort from the patient. The bigger the patient's respiratory effort, the more significant the difference between Pₐₗᵥ and Pₗ, resulting in a less accurate P-V loop.

Therefore, setting the right pressure and volume is especially relevant for patients with damaged lungs which are ventilated with pressure support ventilation (PSV), proportional assist ventilation (PAV, PAV+) and other ventilation modalities such as NAVA where the patient makes an effort.

However, it can be difficult to measure or determine the transpulmonary pressure in a non-invasive way, such that safe ventilation can be offered in a more accurate and convenient way (i.e., without catheters and without avoid or circumvent a patient effort in determining the transpulmonary pressure).

The following discloses certain improvements to overcome these problems and others.

### SUMMARY

In one aspect, a mechanical ventilation device includes at least one electronic controller configured to receive imaging data related to a dimension of a diaphragm of a patient during inspiration and expiration while the patient undergoes mechanical ventilation therapy with an associated mechanical ventilator; calculate a pressure value of a chest of the patient based on at least the imaging data; and when the calculated pressure value does not satisfy an acceptance criterion, at least one of output an alert indicative of the calculated pressure value failing to satisfy the acceptance criterion; and output a recommended adjustment to one or more parameters of the mechanical ventilation therapy delivered to the patient.

In another aspect, a mechanical ventilation method includes, with at least one electronic controller, receiving imaging data related to a dimension of a diaphragm of a patient during inspiration and expiration while the patient undergoes mechanical ventilation therapy with an associated mechanical ventilator; calculating a pressure value of a chest of the patient based on at least the imaging data; and when the calculated pressure does not satisfy an acceptance criterion, at least one of outputting an alert indicative of the calculated pressure value failing to satisfy the acceptance criterion; and outputting a recommended adjustment to one or more parameters of the mechanical ventilation therapy delivered to the patient.

One advantage resides in preventing VILI in patients undergoing mechanical ventilation therapy.

Another advantage resides in determining a transpulmonary pressure of a patient undergoing mechanical ventilation therapy.

Another advantage resides in adjusting settings of a ventilator in delivering mechanical ventilation therapy to a patient based on a calculated a transpulmonary pressure of the patient.

Another advantage resides in automatically adjusting settings of a mechanical ventilator to help wean patients off mechanical ventilation therapy.

Another advantage resides in providing mechanical ventilation therapy without the use of invasive catheters or dedicated ventilation maneuvers for measuring a transpulmonary pressure of the patient.

Another advantage resides in using a detected thickening fraction of the diaphragm to wean a patient off of mechanical ventilation therapy.

Another advantage resides in using ultrasound to non-invasively measure a diaphragm response.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative mechanical ventilation device in accordance with the present disclosure.

FIG. 2 shows an example flow chart of operations suitably performed by the device of FIG. 1.

FIG. 3 shows operations of the flow chart of FIG. 2 as a schematic.

### DETAILED DESCRIPTION

As used herein, the singular form of “a”, “an”, and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

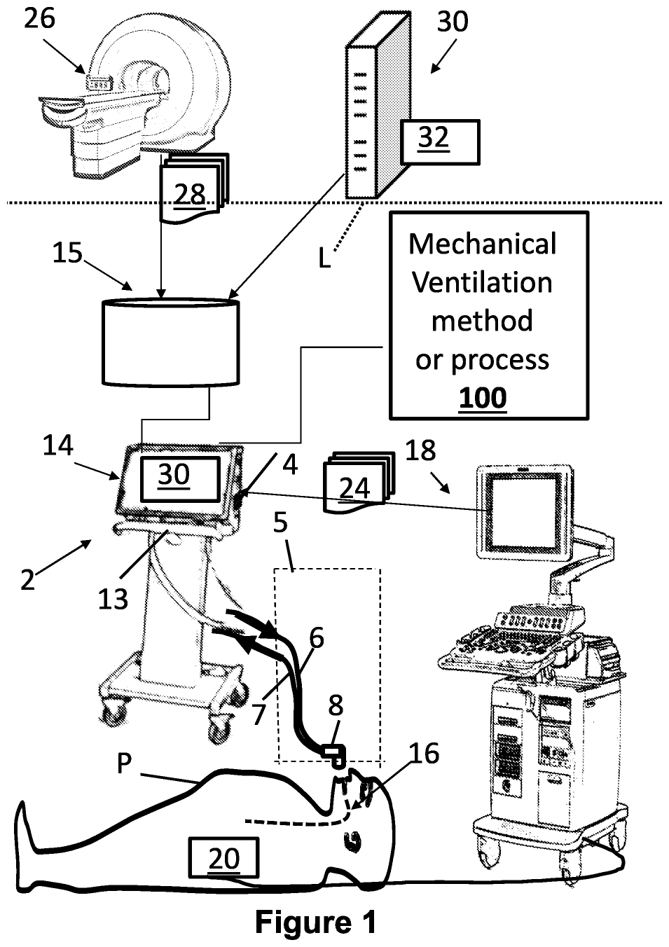

With reference to FIG. 1, a mechanical ventilator 2 for providing ventilation therapy to an associated patient P is shown. As shown in FIG. 1, the mechanical ventilator 2 includes an outlet 4 connectable with a patient breathing circuit 5 to delivery mechanical ventilation to the patient P. The patient breathing circuit 5 includes typical components for a mechanical ventilator, such as an inlet line 6, an optional outlet line 7 (this may be omitted if the ventilator employs a single-limb patient circuit), a connector or port 8 for connecting with an endotracheal tube (ETT) 16, and one or more breathing sensors (not shown), such as a gas flow meter, a pressure sensor, end-tidal carbon dioxide (etCO₂) sensor, and/or so forth. The mechanical ventilator 2 is designed to deliver air, an air-oxygen mixture, or other breathable gas (supply not shown) to the outlet 4 at a programmed pressure and/or flow rate to ventilate the patient via an ETT. The mechanical ventilator 2 also includes an electronic controller 13 (e.g., an electronic processor or a microprocessor), a display device 14, and a non-transitory computer readable medium 15 storing instructions executable by the electronic controller 13.

FIG. 1 diagrammatically illustrates the patient P intubated with an ETT 16 (the lower portion of which is inside the patient P and hence is shown in phantom). The connector or port 8 connects with the ETT 16 to operatively connect the mechanical ventilator 2 to deliver breathable air to the patient P via the ETT 16. The mechanical ventilation provided by the mechanical ventilator 2 via the ETT 16 may be therapeutic for a wide range of conditions, such as various types of pulmonary conditions like emphysema or pneumonia, viral or bacterial infections impacting respiration such as a COVID-19 infection or severe influenza, cardiovascular conditions in which the patient P receives breathable gas enriched with oxygen, or so forth.

FIG. 1 shows the patient P already intubated. That is, FIG. 1 shows the patient after a tracheal intubation has been performed to insert the ETT 16 into the patient. However, to safely perform the tracheal intubation, the anesthesiologist or other qualified medical professional first performs an assessment of the patient P to select the ETT size of the ETT 16, and then inserts an ETT of the selected size into the patient P by a tracheal intubation procedure.

FIG. 1 also shows a medical imaging device 18 (also referred to as an image acquisition device, imaging device, and so forth). As primarily described herein, the medical imaging device 18 comprises an ultrasound (US) medical imaging device 18. In other embodiments, the image acquisition device 18 can be a Computed Tomography (CT) image acquisition device, a C-arm imager, or other X-ray imaging device; Magnetic Resonance (MR) image acquisition device; or a medical imaging device of another modality. As described herein, the medical imaging device 18 is used to acquire ultrasound images of the patient P.

In some embodiments, the medical imaging device 18 can comprise a wearable US imaging device 18. In a more particular example, the medical imaging device 18 includes an ultrasound transducer 20 that is wearable by the patient P (e.g., on the abdomen or chest of the patient P in position to image the diaphragm of the patient, as shown in FIG. 1), such as, for example, a belt or a patch. The US transducer 20 is positioned to acquire US imaging data 24 (i.e., US images) of the diaphragm of the patient P. For example, the US transducer 20 is configured to acquire US imaging data 24 of a diaphragm of the patient P, and more particularly at least a position of the diaphragm of the patient P during inspiration and expiration while the patient P undergoes mechanical ventilation therapy with the mechanical ventilator 2 to determine a diaphragmatic muscle pressure (Pₘᵤₛ) of a chest of the patient P. In another example, the US imaging data 24 can be related to a thickness of the diaphragm of a patient P during inspiration and expiration while the patient P undergoes mechanical ventilation therapy with the mechanical ventilator 2.

In some embodiments, an additional imaging device (e.g., a CT imaging device 26 as shown in FIG. 1) acquires one or more CT images 28 of the patient P. In particular, the CT images 28 can include imaging data of at least a chest wall of the patient P. In another example, the additional imaging device 26 can be an X-ray imaging device configured to acquire X-ray images of the patient P. It should be noted that the CT imaging device 26 may not be located in the same room, or even the same department, as the mechanical ventilator 2. For example, the CT imaging device 26 may be located in a radiology laboratory while the mechanical ventilator 2 may be located in an intensive care unit (ICU), cardiac care unit (CCU), in a hospital room assigned to the patient P, or so forth. This is diagrammatically indicated in FIG. 1 by separator line L.

In some embodiments, a database 30 can store previously-acquired CT (or X-ray) images 28. These images 28 can be retrieved from the database 30 for processing by the electronic controller 13. Similarly to the CT imaging device 26, the database 30 may not be located in the same room, or even the same department, as the mechanical ventilator 2 (or in the same room or department as the CT imaging device 26). As diagrammatically indicated in FIG. 1, the database 30 is disposed on the same side of the separator line L as the CT imaging device 26.

In some embodiments, previously-acquired CT images 28 of the patient P can be used to generate a biomechanical model 32 of thoracic structures (including lungs) and including a chest wall of the patient P. This model 32 can be used as a reference point for analyzing current CT images 28 of the patient P. The model 32 can be stored in the database 30 (or in the non-transitory computer readable medium 15 of the mechanical ventilator 2).

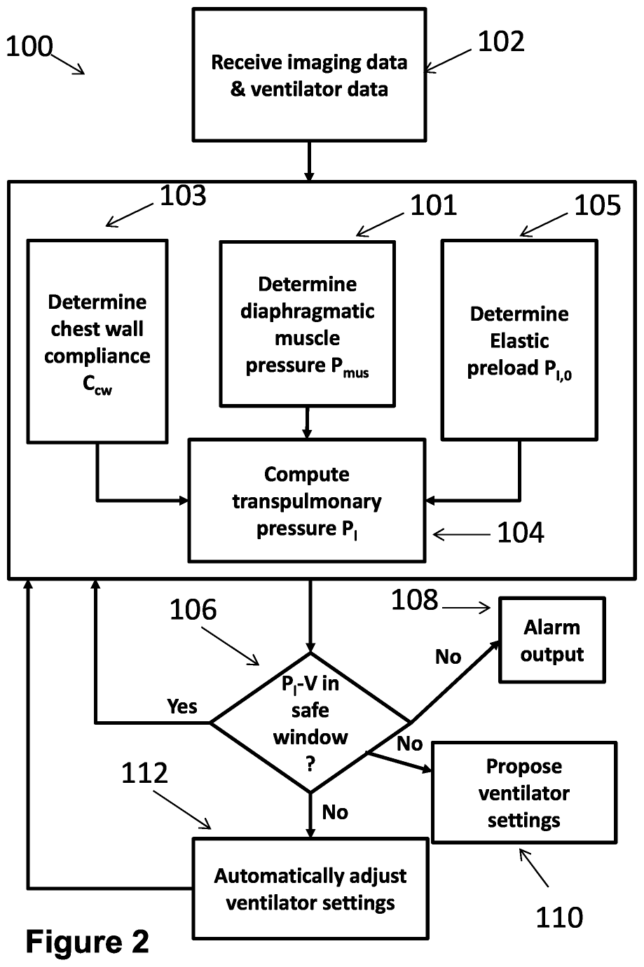

The non-transitory computer readable medium 15 can store instructions executable by the electronic controller 13 to perform a mechanical assistance method or process 100 for monitoring the patient P during mechanical ventilation therapy using the mechanical ventilator 2. With reference to FIG. 2, and with continuing reference to FIG. 1, an illustrative embodiment of a mechanical ventilation 100 is diagrammatically shown as a flowchart. To begin the method 100, the patient P is intubated with the ETT 16 so that mechanical ventilation therapy with the mechanical ventilator 2 can begin, and the ultrasound transducer 20 is attached to the patient so that imaging of the patient P can begin.

At an operation 102, imaging data is received by the electronic controller 13. The imaging data can include data related to a dimension of the diaphragm of a patient P during inspiration and expiration while the patient undergoes mechanical ventilation therapy with the mechanical ventilator 2. The received imaging data includes the US imaging data 24 acquired by the ultrasound transducer 20, and includes a thickness, a thickness change, and/or a position of the diaphragm of the patient P during inspiration and expiration.

In some embodiments, the received imaging data includes the CT images 28 acquired by the CT imaging device 26, and the CT images 28 include at least a chest wall of the patient P. In some embodiments, the received imaging data can comprise previously-acquired images stored in the database 30, and/or can further include retrieving the model 32 of the patient P.

In some embodiments, the electronic controller 13 can also receive data obtained by the mechanical ventilator 2. This ventilator data can include, for example, an airflow during inhalation of the patient P, an airflow pressure in an airway of the patient P during mechanical ventilation therapy, and so forth.

At an operation 104, the electronic controller 13 is configured to calculate a pressure value (i.e., a transpulmonary pressure (Pₗ), a tidal variation in the transpulmonary pressure (DPₗ), and so forth) of the chest of the patient P based on the received data. In one example, the transpulmonary pressure (Pₗ) can be calculated based on the US imaging data 24, the CT images 28, the data received from the mechanical ventilator 2, and so forth. In a particular embodiment, the electronic controller 13 is configured to (i) determine a diaphragmatic muscle pressure (Pₘᵤₛ) from the US imaging data 24 at an operation 101; and (ii) determine a chest wall elastance (E_{cw}) from the CT images 28 (or the previously-acquired images stored in the database 30, or from the biomechanical model 32) at an operation 103. The diaphragmatic muscle pressure (Pₘᵤₛ) and the chest wall elastance (E_{cw}), along with the airway pressure/airflow data from the mechanical ventilator 2, can be used to calculate the transpulmonary pressure (Pₗ) of the chest of the patient P.

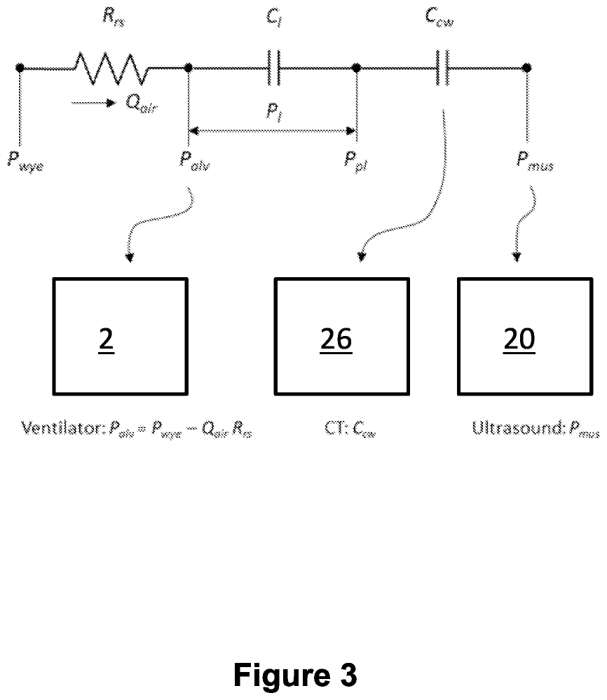

FIG. 3 shows a schematic representation of the operation 104 as a circuit. The circuit represents the respiratory system of the patient P with a total flow resistance in the respiratory system, Rᵣₛ, and two compliances, Cₗ and C_{cw}, respectively of the lungs and the chest wall. Qₐᵢᵣ is the air flow during inhalation. The transpulmonary pressure Pₗ which deforms the lung tissue is the difference between the pleural pressure and the alveolar pressure, Pₗ=Pₐₗᵥ−Pₚₗ. The airway pressure P_{wye} and flow Qₐᵢᵣ are continuously measured by the mechanical ventilator 2, and the flow resistance in the respiratory system Rᵣₛ can be determined with a dedicated ventilator maneuver by the mechanical ventilator 2 (e.g., a least squares process performed by the electronic controller 13).

In FIG. 3, the pressures include (from left to right as shown in FIG. 3): an airway opening pressure (P_{wye}), an alveolar pressure (Pₐₗᵥ), a pleural pressure (Pₚₗ), and an inspiratory muscle pressure (Pₘᵤₛ). The “equation of motion” of the respiratory system in FIG. 3 can be expressed according to Equation (1):

P_{wye}−Pₘᵤₛ=QₐᵢᵣRᵣₛ+V_{T}/Cₗ+V_{T}/C_{cw} (1)

where V_{T} is the tidal volume, which is the integral of the air flow Qₐᵢᵣ. Another equation describing a pressure drop in the airway due to flow resistance can be defined according to Equation 2:

P_{wye}=Pₐₗᵥ+QₐᵢᵣRᵣₛ

By using Equation (2) to solve for P_{wye}, equation (1) can be re-written into equation (3):

Pₐₗᵥ−Pₘᵤₛ=V_{T}/Cₗ+V_{T}/C_{cw} (3)

The alveolar pressure can be determined from the mechanical ventilator 2 according to Equation (2): Pₐₗᵥ=P_{wye}−Qₐᵢᵣ Rᵣₛ.

The term V_{T}/Cₗ in Equation (1) represents an elastic work (i.e., a pressure change) that is needed to deform the lung from its functional residual capacity (FRC) to the volume at the end of inhalation according to Equation (4): Vᵢₙₕₐₗₑ=FRC+V_{T}. This elastic pressure change is the change (i.e., variation) in the transpulmonary pressure, DPₗ=V_{T}/Cₗ during breathing. To determine the absolute value of the transpulmonary pressure, a pre-tension (i.e., an elastic preload) value needs to be determined according to Equation (5): P=P_{l.0}+DPₗ. The pre-tension or preload P_{l.0} is the tension that keeps the lungs inflated at the end of exhalation when there is no action of the respiratory muscles. The pre-tension is the result of the balance between the chest wall which wants to expand (spring-out), and the lungs which want to recoil. This is the reason why the pleural pressure is negative even when the muscle effort is zero in the absence of ventilator support. The nature of this pre-tension becomes clear when there is a puncture in the pleural sac in which case the lung(s) may collapse (i.e., the pneumothorax).

This pre-tension value can be used as a further input to calculate the transpulmonary pressure (Pₗ) of the chest of the patient P, as shown in FIG. 2 at an operation 105. From the functional residual capacity (FRC) and the lung compliance 1/Cₗ=1/Cᵣₛ−1/C_{cw}, it is possible to estimate the elastic preload P_{l.0}=FRC/Cₗ. When the respiratory muscle pressure Pₘᵤₛ=0, the pressures generated by the chest wall and by the lungs are in equilibrium, and the volume V=FRC. When the lungs are collapsed and tensionless, the volume V₀=0. In experiments, it appears that the resting volume RV (i.e., the smallest volume you can get by expiration) is much bigger than the volume that the lung would have in a completely collapsed state (without a ribcage around it). Therefore V₀=0 seems a reasonable assumption or estimation. The relation between pressure and volume is linear. Quantitative analysis of CT images can be used for the FRC measurement.

The term V_{T}/C_{cw} in Equation (1) represents the elastic recoil (i.e., pressure) of the chest wall. During quite tidal breathing this term is negative because of the chest wall tends to “spring out”, in which case the chest wall elasticity works with the diaphragm muscle during a volume expansion of the lungs.

Pₘᵤₛ and C_{cw} can be determined with imaging data of the patient P. Once these parameters are known, and having V_{T} and Pₐₗᵥ continuously available from the mechanical ventilator 2, it is possible to calculate the variation in the transpulmonary pressure on a breath-by-breath basis according to (rewriting Equation (1) as Equation (6)):

DPₗ=V_{T}/Cₗ=Pₐₗᵥ−Pₘᵤₛ−V_{T}/C_{cw} (6)

The inspiratory muscle pressure Pₘᵤₛ can be determined from the US imaging data 24 acquired by the ultrasound transducer 20. The diaphragmatic muscle pressure Pₘᵤₛ can be estimated from the diaphragmatic ultrasound imaging data 24 in different ways. In one example, Eᵣₛ a patient specific biomechanical model 32 is applied to calculate Pₘᵤₛ. The biomechanical model 32 takes as input the diaphragmatic muscle excursion x_{d} obtained from the ultrasound measurements 24 and the pressure from the mechanical ventilator 2, and provides as output the muscle pressure Pₘᵤₛ which is exerted by the diaphragm on the lungs and the chest wall. An advantage of the biomechanical model 32 is that the patient specific geometries are taken into account in the estimation of Pₘᵤₛ. The evaluation of the biomechanical model 32 can be done off-line. The biomechanical model 32 simulates Pₘᵤₛ as a function of the diaphragm excursion x_{d}. The output Pₘᵤₛ as a function of x_{d} is stored in a lookup table (not shown) in the non-transitory computer readable medium 15. The lookup table is then used real time.

In another example, a skeleton muscle model with a force-length relation for muscle fibers can be used (see, e.g., Zhang et al. BioMed Eng OnLine (2016) 15:18, “Biomechanical simulation of thorax deformation using finite element approach”). In such a model, the force generated by a muscle fiber can be determined from its contraction (force-length relationship). The contraction of the diaphragm muscle during an inhalation effort by the patient (thickness and length change) is measured with ultrasound (e.g., the diaphragmatic thickening fraction TFDI and the diaphragm excursion). The muscle force is converted to a muscle pressure by dividing by the projected surface area of the diaphragm, Pₘᵤₛ=Fₘᵤₛ/A_{d} (i.e., a piston model).

The chest wall compliance C_{cw} can be determined, for example, from the CT images 28 acquired by the CT imaging device 26. The chest wall compliance C_{cw} during inspiration can be determined in different ways. In one example, an indirect measurement using a Positive End Expiratory Pressure (PEEP) step method (PSM) can be performed by the electronic controller 13 (see, e.g., Persson, P. et al., 2018, “Evaluation of lung and chest wall mechanics during anaesthesia using the PEEP-step method”, British Journal of Anaesthesia, 120 (4): 860e867 (2018)). The PSM provides the lung compliance Cₗ of a fully sedated patient when Pₘᵤₛ=0. The inverse chest wall compliance is the difference between the inverse respiratory system compliance and the inverse lung compliance according to 1/Cᵣₛ=1/Cₗ+1/C_{cw}. The chest wall compliance is less sensitive to disease than the lung compliance. The PSM procedure can be performed at an early ICU stage when the patient is still fully sedated. The measured C_{cw} can then be used later when there is a respiratory effort.

In another example, a direct measurement process can be performed using a mouthpiece which requires cooperation from the patient (see, e.g., Gideon, E. A. et al., 2021, “The effect of estimating chest wall compliance on the work of breathing during exercise as determined via the modified Campbell diagram”, Am J Physiol Regul Integr Comp Physiol 320: R268-R275). In another example, a look-up table comprising chest wall stiffness values based on patient information such as age and gender can be used (see, e.g., Gideon).

In another example, an algorithm to compute the chest wall stiffness based on a CT scan. A finite element model of the thorax is constructed based on a segmentation of the CT scan (see, e.g., Zhang). Mechanical properties of the structures (intercostal muscle, diaphragm, bone, cartilage, tendons) can be known. The effective chest wall stiffness (inverse compliance) can be calculated by imposing a pressure ΔP in a direction perpendicular to the chest wall (as a boundary condition), and subsequently determine the simulated volume change ΔV from the model output, E_{cw}=1/C_{cw}=ΔP/ΔV. Optionally the finite element model can take into account gravity to evaluate the of tissue (i.e., fat) around the chest or belly, patient position (e.g., prone, supine), or abdominal pressure.

In similar ways the lung compliance can be determined. However, the chest wall compliance is less sensitive to disease properties and the chest wall structure and material properties are less sensitive to person-to-person variations (bone stiffness is known to be less variable than parenchymal tissue). Therefore, it is advantageous to focus on the chest wall compliance.

In another example, a machine learning algorithm to compute the chest wall stiffness (i.e., compliance). The model is trained with imaging (e.g., CT, X-ray, and so forth), data from the mechanical ventilator 2 (V_{T}, Pₐₗᵥ) and catheter data (esophageal pressure, Pₑₛ). The trained model takes as input the imaging and mechanical ventilator data and provides as output Pₑₛ. Subsequently the lung and chest wall compliance can be calculated, subsequently, Cₗ=V_{T}/(Pₐₗᵥ−Pₑₛ) and 1/C_{cw}=1/Cᵣₛ+1/Cₗ.

Referring back to FIGS. 1 and 2, at an operation 106, the electronic controller 13 is configured to determine whether the calculated pressure value (Pₗ, DPₗ) satisfies a predetermined acceptance criterion. For example, the electronic controller 13 can analyze the US images 24 and the calculated pressure value (Pₗ, DPₗ) to determine an effort by the patient P. If the electronic controller 13 determines that the calculated pressure value (Pₗ, DPₗ) does not satisfy the predetermined acceptance criterion, the method 100 proceeds in one or more different ways. In one example embodiment, at an operation 108, an alert indicative of the calculated pressure value (Pₗ, DPₗ) failing to satisfy the acceptance criterion is output. The acceptance criterion may be, for example, that the pressure value (Pₗ, DPₗ) exceeds a threshold value, as dropping below that threshold is considered to be an indication of onset of diaphragm atrophy. This alert output can be done by displaying a message on the display device 14 of the mechanical ventilator 2, thereby indicating to a medical professional that the calculated pressure value (Pₗ, DPₗ) is not satisfactory.

In another example embodiment, at an operation 110, a recommended adjustment to one or more parameters of the mechanical ventilation therapy delivered to the patient P is output. Again, this can be done by displaying a message on the display device 14 of the mechanical ventilator 2, thereby indicating to a medical professional that the calculated pressure value (Pₗ, DPₗ) is not satisfactory.

In a further example embodiment, at an operation 112, the mechanical ventilator 2 is controlled to adjust one or more parameters of the mechanical ventilation therapy delivered to the patient P. If the operation 112 is performed, then the pressure value (Pₗ, DPₗ) can be re-calculated, and this re-calculated pressure value (Pₗ, DPₗ) can be analyzed to determine if the acceptance criterion is met. It will be appreciated that more than one of the operations 108, 110, and 112 can be performed (e.g., the alert can be displayed and the settings of the mechanical ventilator 2 can be adjusted). In some embodiments, the operations 102-106 and at least one of operations 108-112 can be repeated iteratively to provide feedback control of the mechanical ventilator 2 based at least on whether the calculated pressure value (Pₗ DPₗ) satisfies the acceptance criterion.

In a particular embodiment, the display device 14 of the mechanical ventilator 2 is configured to display a pressure-volume (P-V) curve 30 of lungs of the patient P (shown schematically in FIG. 1 as a box) during the mechanical ventilation therapy. The pressure portion of the P-V curve 30 is the calculated pressure value (Pₗ, DPₗ), and the volume portion of the P-V curve 30 is a volume of the lungs of the patient P determined by the CT images 28 (or the biomechanical model 32). In this embodiment, the acceptance criterion is a range of values defined by the displayed P-V curve 30. When the calculated pressure value (Pₗ, DPₗ) is outside of a range of values defined by the displayed P-V curve 30, then one or more of the operations 108, 110, and/or 112 can be performed (e.g., outputting an alert, display a recommended adjustment, or controlling the mechanical ventilator 2 to adjust settings thereof). When the calculated pressure value (Pₗ, DPₗ) is within the range of values, the pressure value (Pₗ, DPₗ) is continuously calculated during the mechanical ventilation therapy to the patient P to ensure the calculated pressure value (Pₗ, DPₗ) is within the range of values.

Modern ventilators can measure the global compliance of the respiratory system. In the measurement procedures a patient effort is avoided (e.g., by taking a measurement at zero flow or during exhalation). However, it is difficult to do this in a reliable and accurate manner since the slopes in the flow curves are high. Consequently, a small misalignment leads to a big error. The diaphragmatic ultrasound imaging data 24 to detect when there is no patient effort, besides looking at the P-V curve 30. To do so, the mechanical ventilator 2 can be synchronized with the ultrasound transducer 20. Pₘᵤₛ (or a surrogate such as TFDI) can be used to plot the P-V curve 30 for display on the display device 14. The compliance of the respiratory system, Cᵣₛ can be determined from the region with neither Pₘᵤₛ nor flow (determined from the volume)

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A mechanical ventilation device comprising at least one electronic controller configured to:
receive imaging data related to a dimension of a diaphragm of a patient during inspiration and expiration while the patient undergoes mechanical ventilation therapy with an associated mechanical ventilator;
calculate a pressure value (Pₗ, DPₗ) of a chest of the patient based on at least the imaging data; and
when the calculated pressure value (Pₗ, DPₗ) does not satisfy an acceptance criterion, at least one of:
output an alert indicative of the calculated pressure value (Pₗ, DPₗ) failing to satisfy the acceptance criterion; and
output a recommended adjustment to one or more parameters of the mechanical ventilation therapy delivered to the patient.

2. The device of claim 1, wherein the at least one electronic controller is configured to calculate the pressure value (Pₗ, DPₗ) by:
determining a diaphragmatic muscle pressure (Pₘᵤₛ) from the received imaging data; and
calculating the pressure value (Pₗ, DPₗ) based on at least the calculated diaphragmatic muscle pressure (Pₘᵤₛ).

3. The device of claim 2, further including:
a wearable ultrasound transducer configured to acquire at least a portion of the imaging data as ultrasound imaging data of at least the diaphragm of the patient.

4. The device of claim 3, wherein the ultrasound imaging data includes at least a position of the diaphragm of the patient during inspiration and expiration while the patient undergoes mechanical ventilation therapy to determine the diaphragmatic muscle pressure (Pₘᵤₛ) and a diaphragm thickness change (TFdi) of the diaphragm.

5. The device of claim 1, wherein the at least one electronic controller is configured to calculate the pressure value (Pₗ, DPₗ) by:
determining a chest wall compliance (C_{cw}) from the received imaging data; and
calculating the pressure value (Pₗ, DPₗ) based on at least the determined chest wall compliance (C_{cw}).

6. The device of claim 5, further including:
an imaging device configured to acquire at least a portion of the imaging data of at least a chest wall of the patient.

7. The device of claim 6, wherein the at least one electronic controller is further configured to:
generate a model of a chest wall of the patient; and
determine the chest wall compliance (C_{cw}) from the received imaging data and the generated model of the chest wall.

8. The device of claim 6, wherein the imaging device comprises one of a computed tomography (CT) imaging device or an X-ray imaging device.

9. The device of claim 5, further including:
a database storing previously-acquired computed tomography (CT) imaging data and/or previously-acquired X-ray imaging data of at least the chest wall of the patient.

10. The device of claim 1, wherein the at least one electronic controller is further configured to:
receive, from the associated mechanical ventilator, at least one of an airflow during inhalation of the patient and an airflow pressure in an airway of the patient during mechanical ventilation therapy; and
calculate the pressure value (Pₗ, DPₗ) further based on the airflow and/or the airflow pressure.

11. The device of claim 10, further including:
a mechanical ventilator configured to deliver mechanical ventilation therapy to the patient;
wherein the mechanical ventilator is configured to measure the at least one of an airflow during inhalation of the patient and an airflow pressure in an airway of the patient during mechanical ventilation therapy.

12. The device of claim 11, wherein the mechanical ventilator includes a display device, and the at least one electronic controller is further configured to:
control the display device to display a pressure-volume curve of lungs of the patient during the mechanical ventilation therapy; and at least one of:
output the alert when the calculated pressure value (Pₗ, DPₗ) is outside of a range of values defined by the displayed pressure-volume curve; and
output a recommended adjustment to one or more parameters of the mechanical ventilation therapy delivered to the patient.

13. The device of claim 1, wherein the at least one electronic controller is further configured to calculate the pressure value (Pₗ, DPₗ) by:
calculating, from the received imaging data, a pre-tension value of lungs of the patient at the end of exhalation when there is no action by respiratory muscles of the patient;
calculating the pressure value (Pₗ, DPₗ) further based on the calculated pre-tension value.

14. The device of claim 11, wherein the at least one electronic controller configured to is configured to:
control the mechanical ventilator to adjust one or more parameters of the mechanical ventilation therapy delivered to the patient based on the calculated pressure value (Pₗ, DPₗ).

15. A mechanical ventilation method comprising, with at least one electronic controller:
receiving imaging data related to a dimension of a diaphragm of a patient during inspiration and expiration while the patient undergoes mechanical ventilation therapy with an associated mechanical ventilator;
calculating a pressure value (Pₗ, DPₗ) of a chest of the patient based on at least the imaging data; and
when the calculated pressure (Pₗ, DPₗ) does not satisfy an acceptance criterion, at least one of:
outputting an alert indicative of the calculated pressure value (Pₗ, DPₗ) failing to satisfy the acceptance criterion; and
outputting a recommended adjustment to one or more parameters of the mechanical ventilation therapy delivered to the patient.
